SEQUENCE LISTING (5 sequences)

SEQ ID NO: 1 - DNA, 1740 bp, Oryza sativa
  source 1..1740: mol_type = other DNA
atgcctgaggcaaatgcgggtgggcgggcggcgccggcggcggagcagcggagcaagaacacaacagagatggatttcttcagtgaatatggtgactccagccgatacaaaattcaagaaatcgttggtaaaggaagttatggagttgtttgttcagctattgaccaacataccggtgacaaagtagcaatcaagaaaatacacaatatctttgagcatctatctgatgctgctaggatcctccgtgagatcaaacttctccggcttttacggcatcctgatattgtcgagatcaaacatataatgttacctccatctagaagggacttcaaagatatttatgttgtcttcgaactgatggatacagacctccaccaggttattaaggctaatgatgacttaacgaaggaacatcatcagttctttctgtatcaaatgcttcgagcattgaaatatatccatactgctaatgtttatcatcgtgatttaaagcccaaaaatatattagcaaatgctaactgtaagctcaagatatgtgattttggactagcaagagttgcatttaatgacactcctacaacagttttctggacggactacgttgcaactagatggtacagggctcctgagctgtgtgggtctttcttttctaagtattcaccagctatagatacatggagtattggttgcatttttgcggagattttgactggaaaacctttgttccctggtaaaaatgtggttcatcaattggacttgatgactgatctcttgggtacaccatcaatggatgctatttcaaggattcggaatgacaaggcaaggaggtatctgagcagcatgaggaggaagcagccagtacctttttcagaaaagttcccaaatgtagatcctttggcactcaagctcttacaaaggcttctagcatttgatccgaaggatcgacctactgcagaagaggcgttggctgatccatattttaaaggccttgcaaaagtggagagagaaccatcatgccaaccaatttcaaaaatggagtttgagtttgagcgaagaaaggttaccaaagatgatatcaaggaacttatattccgtgagatattagagtatcatcctcagcttctgaaggattacatgaatggctccgaaaacacgagctttctatatccaagtgctgttgataatttccggaggcaatttgccatcttagaggaaaacggaggaaagagtggtgcactagataggaagcatgtttctcttccaagggctacaacagttcactctacatcaattcctccaaatgaaggcctagatgcaacatcccaagttactcaaaggatcccaacagctagaccaggaagaacggttggtccggtattaccattcgagaatccaggcgccgcagatccgcacagcgcacggagggtggtgaggaatccgatggttcctccagcagctgccaacaagtcaggatacagctacaacctaaagtcagactactctgataggcaacatcaggaagagcttgagaaagatcgtgtgcagtacaggcctgcacaacacttgatggatgctaaagttgctccagatacagccccggacatacggtcttcacaatactactttacgaggagtgctcccagaactgatctaacagacagggctgcactccaggggagcatgctatacggcattgctccgttcaacggcatcgcagcagttgctggtggatacagcaag

SEQ ID NO: 2 - DNA, 24 bp, synthetic construct
  source 1..24: mol_type = other DNA; note = Sequence of forward primer OsMAPK7-OsU6a-F for PCR
gccgtacggcattgctccgttcaa

SEQ ID NO: 3 - DNA, 24 bp, synthetic construct
  source 1..24: mol_type = other DNA; note = Sequence of reverse primer OsMAPK7-OsU6a-F for PCR
aaacttgaacggagcaatgccgta

SEQ ID NO: 4 - DNA, 24 bp, synthetic construct
  source 1..24: mol_type = other DNA; note = Sequence of forward primer OsMAPK7-OsU6b-F for PCR
gttgactgctgcgatgccgttgaa

SEQ ID NO: 5 - DNA, 24 bp, synthetic construct
  source 1..24: mol_type = other DNA; note = Sequence of reverse primer OsMAPK7-OsU6b-F for PCR
aaacttcaacggcatcgcagcagt

(19) United States
(12) Patent Application Publication
(10) Pub. No.: US 2025/0163447 A1
(43) Pub. Date: May 22, 2025

(54) USE OF OsMAPK7 GENE IN CONVERTING SUBMERGENCE TOLERANCE OF RICE

(71) Applicant: GUANGXI UNIVERSITY, Nanning (CN)

(72) Inventors: Jianxiong LI, Nanning (CN); Yao WAN, Nanning (CN); Kaichong TENG, Nanning (CN); Fengling LIN, Nanning (CN)

(73) Assignee: GUANGXI UNIVERSITY, Nanning (CN)

(21) Appl. No.: 18/512,762

(22) Filed: Nov. 17, 2023

(51) Int. Cl. C12N 15/82

(52) U.S. Cl. CPC C12N 15/8271; C12N 15/8218; C12N 2310/20

(57) ABSTRACT

The present disclosure provides use of an OsMAPK7 gene in transforming non submergence tolerance rice to submergence tolerance rice, and belongs to the technical field of gene function. In the present disclosure, the OsMAPK7 gene has a nucleotide sequence shown in SEQ ID NO: 1. It is found that knocking out the OsMAPK7 gene in rice can transform ordinary cultivated rice that is intolerant to waterlogging into cultivated rice that is tolerant to waterlogging. This process can improve a germination rate of rice seeds, promote root system growth of rice seedlings, and improve a survival rate of rice seedlings under waterlogging. Therefore, this process solves a bottleneck faced by direct-seeded rice production, thereby ensuring the planting area and yield of rice production. The present disclosure further provides a method for converting submergence tolerance of rice, which shows an important utilization value in rice production.

### TECHNICAL FIELD

The present disclosure relates to the technical field of gene function, and specifically relates to use of an OsMAPK7 gene in converting submergence tolerance of rice.

### REFERENCE TO SEQUENCE LISTING

A computer readable XML file entitled “GWP20231008189-Sequence listing”, which was created on Nov. 10, 2023, with a file size of about 7,674 bytes, contains the sequence listing for this application, has been filed with this application, and is hereby incorporated by reference in its entirety.

### BACKGROUND

Rice is an important food crop. Natural disasters have a great impact on rice production. For example, floods generally occur in tropical and subtropical regions. Flooding caused by continuous rain during a rice sowing period can seriously affect the germination rate of rice and lead to a reduction in rice production. As young rural laborers move to cities, rice production faces labor shortages. For this reason, when planting rice in many rural areas, direct seeding is adopted instead of traditional methods of raising seedlings and then transplanting rice seedlings. Large-scale of using rice direct seeding can save a lot of labor and alleviate the contradiction of seasonal labor shortage. This is of great significance to the lightness, specialization, and scale of rice production, and can ensure the production area and yield of rice, thus showing broad prospects for rice production. During direct seeding of rice, the rice seeds falling on waterlogged plots may not germinate or have a reduced germination rate if the rice fields are not leveled and some plots are waterlogged. In some cases, heavy rains cause waterlogging in the rice fields during rice germination, leading to lower germination rate and greatly affecting the growth of rice seedlings. In addition, direct-seeded rice suffers from serious weed damage and must be chemically weeded, which can increase agricultural input and labor costs and also cause an impact on environmental safety. Moreover, the direct-seeded rice has root systems that do not penetrate deeply into the soil and are prone to lodging. If the direct-seeded rice is a waterlogging-tolerant rice variety, the germination of rice seeds is not affected even being sown in a waterlogged area or being kept in the rice flooding fields for a long period of time after the direct-seeding. Thus, submergence tolerance rice is much needed. Using submergence tolerance rice in direct rice seeding does not affect the germination of rice seeds, and inhibits the growth of weeds to reduce their damage, thereby eliminating the requirement for chemical weeding to achieve environmental friendliness. In addition, if submergence-tolerant rice variety has strong root systems under waterlogging, it can also avoid the easy lodging of direct-seeded rice. Taken together, the finding of a gene that can convert non submergence rice to submergence rice will contribute a lot to rice production area and yield.

### SUMMARY

In view of this, an objective of the present disclosure is to provide use of an OsMAPK7 gene in converting the non-submergence rice to submergence tolerance rice. In the present disclosure, knocking out the OsMAPK7 gene can improve a germination rate of rice seeds and promote the root system growth of rice seedlings under submergence.

To achieve the above objective, the present disclosure provides the following technical solutions:

The present disclosure provides use of a OsMAPK7 gene in converting submergence tolerance of rice, where the OsMAPK7 gene has a nucleotide sequence shown in SEQ ID NO: 1.

Preferably, the rice includes Japonica rice and indica rice.

Preferably, the use includes improving a germination rate of a rice seed under submergence.

Preferably, the use includes promoting growth of a root system of a rice seedling under submergence.

The present disclosure further provides a method for converting non-submergence rice to submergence tolerance rice, including: knocking out an OsMAPK7 gene.

Preferably, the knocking out is conducted by a clustered regularly interspaced short palindromic repeat/CRISPR-associated protein 9 (CRISPR/Cas9) technology.

Compared with the prior art, the technical solutions of the present disclosure have the following beneficial effects:

OsMAPK7 is a common gene in cultivated rice. In the present disclosure, it was discovered for the first time that the OsMAPK7 gene is related to the submergence tolerance of rice. It is also found that knocking out the OsMAPK7 gene in rice can transform ordinary cultivated rice that is intolerant to submergence into cultivated rice that is tolerant to submergence. This transformation also can improve a germination rate of rice seeds, promote root system growth of rice seedlings, and improve a survival rate of rice seedlings under submergence. Therefore, this process solves a bottleneck faced by direct-seeded rice production, thereby ensuring the planting area and yield of rice production. Accordingly, this gene exhibits an important utilization value in rice production.

### BRIEF DESCRIPTION OF THE DRAWINGS

FIG. 1 shows sequencing results of some CRISPR/Cas9-OsMAPK7 knockout rice lines;

FIG. 2 shows germination and seedling growth of Zhonghua 11 (ZH11), OsMAPK7-Cas9, and OsMAPK7-OE seeds under waterlogging in test tubes for 14 d; and

FIG. 3 shows growth of seeds for Yasi 881 and Huanghuazhan and their OsMAPK7-Cas knockout plants after 14 d of field waterlogging.

### DETAILED DESCRIPTION OF THE EMBODIMENTS

The present disclosure provides use of an OsMAPK7 gene in converting submergence tolerance of rice. The OsMAPK7 gene is a gene commonly found in cultivated rice and has a nucleotide sequence shown in SEQ ID NO: 1.

In the present disclosure, the rice includes Japonica rice and indica rice. The Japonica rice is preferably Japonica rice ZH11, and the indica rice includes preferably indica rice Yasi 881 and indica rice Huanghuazhan.

In the present disclosure, the use includes: improving a germination rate of a rice seed under waterlogging and promoting growth of a root system of a rice seedling under waterlogging.

The present disclosure further provides a method for converting submergence tolerance of rice, including: knocking out OsMAPK7 gene. The knocking out is conducted preferably by a CRISPR/Cas9 technology.

As an optional example, a process of knocking out the OsMAPK7 gene using the CRISPR/Cas9 technology includes: designing a target primer corresponding to the OsMAPK7 gene; ligating the target primer to an sgRNA expression cassette by overlapping PCR to construct a modified sgRNA expression cassette, and cloning the modified sgRNA expression cassette into a plant gene editing vector; using a resulting ligation product to construct a transgenic rice by Agrobacterium transformation to obtain a OsMAPK7 gene-knockout rice line.

The technical solutions of the present disclosure will be clearly and completely described below with reference to the examples of the present disclosure. Apparently, the described examples are merely a part rather than all of the examples of the present disclosure. All other embodiments obtained by those skilled in the art based on the embodiments of the present disclosure without creative efforts shall fall within the protection scope of the present disclosure.

In this example, the plant gene editing vector is pYLCRISPR/Cas9Pubi-H, promoters are U6a and U6b, and methods are Overlapping PCR and Golden Gate cloning.

### Example 1

A cDNA sequence of the OsMAPK7 gene was obtained through an NCBI website (https://www.ncbi.nlm.nih.gov); a target primer was designed by CRISPR-GE: http://skl.scau.edu.cn/home/for a gene editing vector. Based on a design principles of efficient target sites such as a GC content of 45% to 70% and off-target <0.6, the target sites used promoters U6a and U6b, and a multiple cloning site where the OsMAPK7 gene was inserted was Bsa I.

The target primer was ligated to an sgRNA expression cassette by overlapping PCR to construct a modified sgRNA expression cassette, including two rounds of PCR reactions:

A first round of PCR reaction: a target was introduced downstream of the U6 promoter and upstream of an sgRNA sequence.

PCR for a reaction in first step was as follows:

TABLE 1
PCR reaction system in first step

| Component | Content | |
|---|---|---|
| 2 × Phanta Max Buffer | 7.5 | μL |
| 10 mmol/L dNTPs Mix | 0.25 | μL |
| Phanta Max Super-Fidelity DNA Polymerase | 0.3 | μL |
| YLgRNA-U6 | 0.5 | μL |
| 10 μmol/L U-F and U#-T# (reaction 1) | Each 0.3 μL | |
| 10 μmol/L gR-T# and gR-RL (reaction 2) | Each 0.3 μL | |
| ddH₂O | 5.85 | μL |
| Total | 15 | μL |

TABLE 2
PCR reaction program in first step

| Step | Temperature | Time (s) | Cycle |
|---|---|---|---|
| Initial denaturation | 95 | 30 | 1× |
| Denaturation | 95 | 15 | |
| Annealing | 58 | 15 | 26× |
| Extension | 72 | 20 | |
| Final extension | 72 | 60 | 1× |

A second round of PCR reaction: the promoter, target, and sgRNA were constructed into a complete expression cassette. A product of the first round of PCR was diluted tenfold and used in the second round of PCR.

TABLE 3
PCR reaction system in second step

| Component | Content | |
|---|---|---|
| ddH₂O | 11.8 | μL |
| 2 × Phanta Max Buffer | 15 | μL |
| dNTP Mix (10 mM each) | 0.6 | μL |
| Pps-R/Pps-2 | 0.5 | μL |
| Pgs-2/Pgs-L* | 0.5 | μL |
| Phanta Max Super-Fidelity DNA Polymerase | 0.6 | μL |
| 10 × mixing with product in first step | 1 | μL |
| Total | 30 | μL |

TABLE 4
PCR reaction program in second step

| Step | Temperature (° C.) | Time (s) | Cycle |
|---|---|---|---|
| Initial denaturation | 95 | 30 | 1× |
| Denaturation | 95 | 15 | |
| Annealing | 58 | 15 | 30× |
| Extension | 72 | 20 | |
| Final extension | 72 | 60 | 1× |

Product purification: a PCR product from the previous step was purified to remove proteins, oligonucleotides, etc., to obtain a relatively pure sgRNA expression cassette for subsequent experiments. The purification of PCR product in this experiment was completed using Vazyme's FastPure Gel DNA Extraction Mini Kit, including:
(1) ddH₂O was added into the product of the previous step to reach 100 μL, then 500 μL of Buffer GDP was added and mixed by inverting; a resulting mixture was transferred to an adsorption column, the adsorption column was placed in a collection tube, and then centrifuged in a centrifuge tube of a centrifuge at 13,400×g for 60 s;
(2) the centrifuge tube was taken out, a filtrate was discarded, 700 μL of Buffer GW with absolute ethanol were added to the adsorption column, and the collection tube was placed into a centrifuge to allow centrifugation at 13,400×g for 60 s;
(3) step (2) was repeated;
(4) the centrifuge tube was taken out, a filtrate was discarded, the adsorption column was put back into the collection tube, and the collection tube was placed in a centrifuge to allow centrifugation at 13,400×g for 2 min; and
(5) the centrifuge tube was taken out, the adsorption column was put into a new 1.5 mL centrifuge tube, 30 μL of 55° C.-preheated ddH₂O was added to a membrane center of the adsorption column, and allowed to stand at room temperature for 2 min; the collection tube was put into a centrifuge and centrifuged at 13,400×g for 1 min, and a filtrate obtained was a purified product.

Cloning of the sgRNA expression cassette into a pYLCRISPR/Cas9Pubi-H vector: the purified product in the previous step and the pYLCRISPR/Cas9Pubi-H vector were digested with Bsa I and then ligated with a T4 DNA ligase, such that the sgRNA expression cassette was inserted into the pYLCRISPR/Cas9Pubi-H vector by cutting while ligating using the “Golden Gate” cloning. This reaction was conducted on a PCR machine and a hot lid was closed. The detailed reaction system and procedures were as follows:

TABLE 5
“Golden Gate” cloning system

| Component | Content | |
|---|---|---|
| pYLCRISPR/Cas9Pubi-H (80 ng/μL) | 1 | μL |
| Purified sgRNA expression cassette mixture | 1 | μL |
| 10 × Cutsmart Buffer | 1.5 | μL |
| 10 × T₄ DNA ligase buffer | 1.5 | μL |
| Bsa I-HFv2 (20 U/μL) | 0.5 | μL |
| T₄ DNA ligase (400 U/μL) | 0.2 | μL |
| ddH₂O | 9.3 | μL |
| Total | 15 | μL |

TABLE 6
“Golden Gate” cloning procedures

| Step | Temperature (° C.) | Time (s) | Cycle |
|---|---|---|---|
| First step | 37 | 5 | |
| | 10 | 5 | 15× |
| | 20 | 5 | |
| Second step | 37 | 5 | 1× |

The primers were as follows:

OsMAPK7-OsU6a-F:
(SEQ ID NO: 2)
GCCGTACGGCATTGCTCCGTTCAA;
OsMAPK7-OsU6a-R:
(SEQ ID NO: 3)
AAACTTGAACGGAGCAATGCCGTA;
OsMAPK7-OsU6b-F:
(SEQ ID NO: 4)
GTTGACTGCTGCGATGCCGTTGAA;
OsMAPK7-OsU6b-R:
(SEQ ID NO: 5)
AAACTTCAACGGCATCGCAGCAGT.

A ligation product from the above steps was transferred into DH5α competent E. coli using a heat shock method, spread on an LB screening plate containing 25 μg/mL kana, and incubated in a 37° C. incubator for 12 h to 16 h until positive plaques appeared. A plasmid of the positive clone was transferred to Agrobacterium, and rice callus was transformed by the Agrobacterium.

Construction of transgenic rice: calli were induced from mature seeds of Zhonghua 11 (Japonica rice), Huanghuazhan (indica rice), and Yasi 881 (indica rice), and the calli were subcultured after 21 d; the subculture was conducted once every 10 d, and embryogenic callus in good conditions after 2 times of subculture was selected for infection with Agrobacterium EHA105 vector; after 3 d of co-culture, a resistant callus was screened, and the regeneration of transgenic plants was conducted by screening using 50 mg/L hygromycin medium every 2 weeks; the resistant calli in good conditions were selected for 2 weeks of pre-differentiation culture, and then 2 weeks of differentiation culture; obtained differentiated seedlings were transferred to a rooting medium to obtain the transgenic plants. After PCR detection of the hygromycin gene Hpt, and sequencing of the target gene after PCR reaction, CRISPR-Cas9-OsMAPK7 knockout transgenic positive plants were obtained. FIG. 1 showed sequencing results of some CRISPR/Cas9-OsMAPK7 knockout rice lines, indicating that the OsMAPK7 gene was edited.

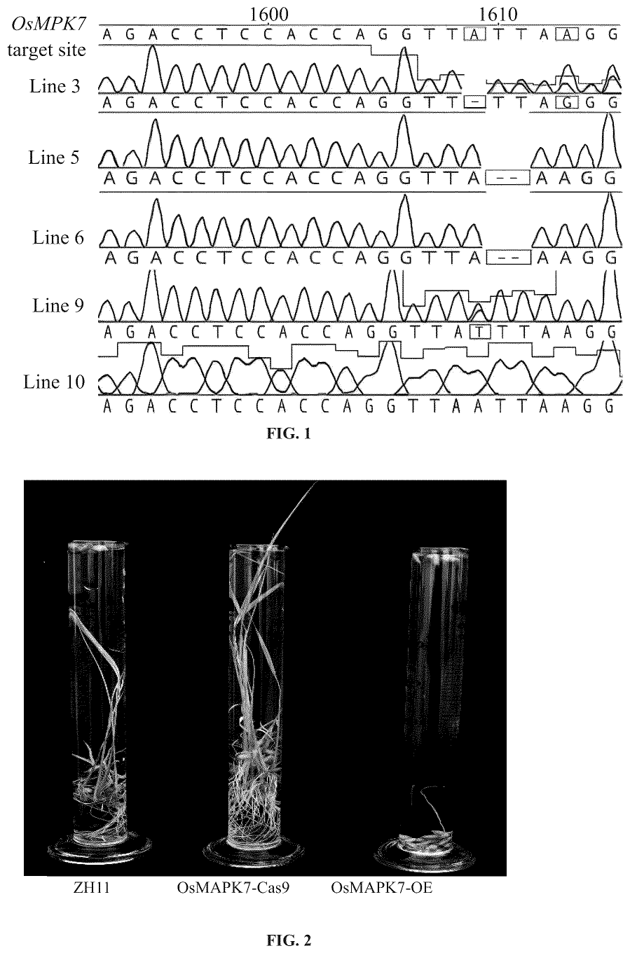

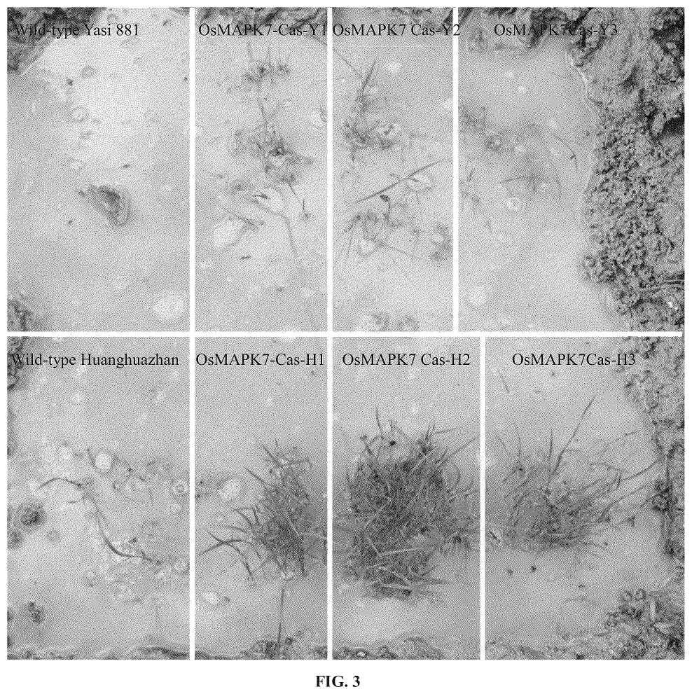

The seeds from transgenic plants with OsMAPK7 knockout by CRISPR-Cas9 were subjected to submergence tolerance experiments during the germination period under test tube and field. The results were shown in FIG. 2 and FIG. 3.

FIG. 2 showed germination and seedling growth of Zhonghua 11 (ZH11), OsMAPK7 knockout (OsMAPK7-Cas9), and OsMAPK7 overexpression (OsMAPK7-OE) seeds under waterlogging in test tubes for 14 d. It was seen from this figure that the seeds of OsMAPK7-Cas9 plants could germinate normally, their seedlings could elongate and emerge out of the water, and their root systems were also longer than those of wild-type ZH11. This indicated that knocking out OsMAPK7 in Japonica rice ZH11 could transform the ZH11 that was intolerant to waterlogging into the ZH11 that was tolerant to waterlogging.

FIG. 3 showed the germination and growth of seeds from wild-type Yasi 881/wild-type Huanghuazhan (indica varieties) and their respective OsMAPK7-knockout (OsMAPK7-Cas9) plants after being waterlogged in the field for 14 d. In this figure, OsMAPK7-Cas-Y1, OsMAPK7 Cas-Y2, and OsMAPK7Cas-Y3 represented three OsMAPK7-knockout lines of Yasi 881, respectively; while OsMAPK7-Cas-H1, OsMAPK7 Cas-H2, and OsMAPK7Cas-H3 represented three OsMAPK7-knockout lines of Huanghuazhan, respectively. When being sown under waterlogging in the field, the wild-type Yasi 881 seeds did not germinate, and the Huanghuazhan seeds had an extremely low germination rate (germination rate 3%). The transgenic seeds with OsMAPK7 gene knockout had an extremely high germination rate under waterlogging (OsMAPK7 was knocked out in Yasi 881, and an average germination rate was 75%), or were completely germinated (OsMAPK7 was knocked out in Huanghuazhan, and an average germination rate was 90%). This proved that OsMAPK7 knockout (OsMAPK7-Cas9) plants showed submergence tolerance.

The above experimental results show that in indica rice and Japonica rice varieties, the seeds from transgenic plants with the OsMAPK7 gene knockout have better germination rates, longer root systems, and seedlings that can elongate out of the water than those of the wild-type seeds under long-term waterlogging.

The above descriptions are merely preferred implementations of the present disclosure. It should be noted that a person of ordinary skill in the art may further make several improvements and modifications without departing from the principle of the present disclosure, but such improvements and modifications should be deemed as falling within the protection scope of the present disclosure.

What is claimed is:

1. A method for producing submergence tolerance rice, comprising: knocking out an OsMAPK7 gene using clustered regularly interspaced short palindromic repeat/CRISPR-associated protein 9 (CRISPR/Cas9).

2. The method according to claim 1, wherein the OsMAPK7 gene has a nucleotide sequence shown in SEQ ID NO: 1.

3. The method according to claim 1, wherein the rice comprises Japonica rice and indica rice.

4. The method according to claim 1, comprising improving a germination rate of a rice seed under waterlogging.

5. The method according to claim 1, comprising promoting growth of a root system of a rice seedling under waterlogging.

6. A method for converting submergence tolerance of rice, comprising: knocking out an OsMAPK7 gene.

7. The method according to claim 6, wherein the knocking out is conducted using clustered regularly interspaced short palindromic repeat/CRISPR-associated protein 9 (CRISPR/Cas9).
